SEQUENCE LISTING (9 sequences)

SEQ ID NO: 1 - DNA, 14719 bp, synthetic construct
  source 1..14719: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgatgatggcttctaaggatgctacatcatctgtggatggagctagtggagctggtcaattggttccagaggttaatgcttctgaccctcttgctatggatcctgtagcaggttcttccacagcagttgctactgctggacaagttaatcctattgatccatggataattaacaactttgtgcaagccccccaaggtgaattcactatttccccaaacaacaccccaggtgatgttttgtttgatttgagtttgggtccccatcttaatcctttcttgctccatctctcacaaatgtataatggttgggttggtaacatgagagttaggattatgcttgctggtaatgcctttactgctggtaagataatagtttcttgcataccccctggttttggttcacataatcttactatagcacaagcaactctctttcctcatgtgattgctgatgttaggactcttgaccccattgaggtgcctttggaagatgttaggaatgttctctttcataacaacgatagaaatcaacaaaccatgaggcttgtgtgcatgctctacacccccttgaggactggtggtggtactggtgattcttttgtagttgcaggaagggttatgacttgcccaagtcctgattttaatttcttgtttttagtccctcctacagtggagcaaaaaaccaggcccttcacactcccaaatctcccattgagttctctctctaactcaagagcccctctcccaattagtagtatgggcatttccccagacaatgtccaaagtgtgcaattccaaaatggtaggtgtactcttgatggaagacttgttggcaccaccccagtaagcttgtcacatgttgccaagataagaggtacctccaatggcactgtgatcaaccttactgaattggatggcacaccctttcacccttttgagggccctgcccccattggatttccagatcttggtggttgtgattggcatatcaatatgacacaatttggccattctagccaaacccaatatgatgtcgacaccacccctgacacttttgtcccccatcttggttcaattcaagcaaatggcattggaagtggtaattatgttggtgttctttcttggatttcccccccatcacacccatctggctcccaagttgacctttggaagatccccaattatggatcaagtattactgaggcaacacatcttgccccttctgtatacccccctggttttggagaggtattggtctttttcatgtcaaaaatgccaggtcctggcgcttataatttgccatgtctcttaccacaagagtacatttcacatcttgctagcgagcaagcccctactgtaggtgaggctgccctgctccactatgttgaccctgatactggtaggaatcttggagaattcaaagcataccctgatggtttcctcacttgtgtccccaatggtgctagcagcggtccacaacaactgccaatcaatggtgtctttgtctttgtttcatgggtgtcaagattttatcaattaaagcctgtgggaactgcctctagcgcaagaggtaggcttggtcttaggaggtaagagctcgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgctcatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcatgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 2 - DNA, 5020 bp, synthetic construct
  source 1..5020: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatgaattcttttggtctcactagggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgggcccaagcgcaagcaaaaattttagactgcaaagcaaatacgtgttcctgacctaccccaaatgctccagccagagggatgacctgttccagttcctgtgggagaagctgactcccttcctgatcttcttcctgggcgtggcctccgaactgcatcaggacggaaccacacattaccatgctctgattcagctggacaaaaaaccatgcattagagatccatcctttttcgacttcgaagggaaccatcctaatatccagcccgctagaaattccaagcaggtcctggactacatttccaaggacggagatattaaaaccagaggagatttcagggatcataaagtgagcccaaggaagtccgacgccagatggaggaccatcatccagacagccactagcaaggaagagtacctggacatgattaaggaagaatttcctcacgaatgggctacaaaactgcaatggctggagtacagcgccaataaactgtttcccccccagcctgagcagtacgtgtccccattcactgaaagcgacctgaggtgccatgaggacctgcacaactggagggaaactcatctgtaccatgacgaagggaggaccggagtgaggcacccctccctgtacatctgcggacctactagaaccggcaaaactacctgggctagatccctgggcagacataactactggaatggcaccattgatttcaccaactacgacgagcatgctacatacaacatcattgacgacatccctttcaaatttgtgccactgtggaagcaactgatcggctgccaatccgactttacagtgaaccccaaatacgggaagaaaaaaaagattaaaggcggcatcccaagcattatcctgtgcaatcccgatgaggattggatgctgtccatgacaagccagcagaaggactactttgaggacaattgtgtgacccattatatgtgtgacggggaaaccttctttgccagggagtccagctcccattgagagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttagctagagaccaaaaggatccatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattgttatccgctcacaattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 3 - DNA, 4894 bp, synthetic construct
  source 1..4894: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatgaattcttttggtctcactagggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggggccttccgcaagcaagaactttaggctgcaatccaaatatgtcttcctgacctatccaaagtgtagcagccagagggatgacctgttccagttcctgtgggagaagctgactccatttctgatcttctttctgggagtggcctccgagctgcaccaggacggaactacacactaccatgccctgattcagctggacaagaagccatgtatcagggacccctccttctttgatttcgaaggaaatcatcccaacatccagcctgcaagaaactccaaacaggtgctggattacatcagcaaagacggggatatcaaaacaagaggcgattttagggaccacaaggtgagccccaggaagtccgacgccaggtggaggaccattatccagacagccacaagcaaggaggagtacctggatatgatcaaagaggaatttccccacgaatgggcaactaaactgcaatggctggagtatagcgctaacaagctgttcccaccacagccagaacagtatgtctccccattcacagaaagcgacctgagatgtcatgaggatctgcataactggagggagactcatctgtatcatgtgagcatcgacgcttacaccttcatccatcccgtgagctacgaccaggcccaatccgacctggaatggatggctgatctgaccagaatgagagaaggcctggggagcgatactcctgccagcacatccgccgatcagctggtgcctgagaggccccctggactggaggtgagcggagatacaaccacagggactggacctagcaccagcccaacaaccatgaatacacctcccattatcagcagcaccacctccccaagcagcagctcccattgtgggtccaactgagagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttagctagagaccaaaaggatccatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattgttatccgctcacaattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 4 - DNA, 16785 bp, synthetic construct
  source 1..16785: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagctagggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttagctagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatcggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaggatccccgggtaccgagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 5 - DNA, 10768 bp, synthetic construct
  source 1..10768: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctgggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcactagggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttagctgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaaggctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgcctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 6 - DNA, 352 bp, Bean yellow dwarf virus
  source 1..352: mol_type = genomic DNA
agaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacg

SEQ ID NO: 7 - DNA, 214 bp, Bean yellow dwarf virus
  source 1..214: mol_type = genomic DNA
gagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaag

SEQ ID NO: 8 - DNA, 237 bp, synthetic construct
  source 1..237: mol_type = other DNA
atggagattaccgtgaagacccttttgcgtatgaagatcgccctcgaggttgaaccctcggatacgatagaaaatgtaaaggccaagatccaggataaggaaggaattcctcctgatcagcagcggctgatctttcgtggcttgctcctggaagatggacgtactttgtctgactacaatattcaaaagggctctactcttactcttggcttggttcttcgtgataatgctgtttaa

SEQ ID NO: 9 - protein, 78 aa, synthetic construct
MEITVKTLLRMKIALEVEPSDTIENVKAKIQDKEGIPPDQQRLIFRGLLLEDGRTLSDYNIQKGSTLTLGLVLRDNAV

(19) United States
(12) Patent Application Publication
(10) Pub. No.: US 2026/0357280 A1
(43) Pub. Date: Nov. 12, 2026

(54) A METHOD FOR PRODUCTION OF SELF-REPLICATING, NUCLEIC ACID-LOADED, VIRUS-LIKE PARTICLES (VLP-NA) AND THE USES THEREOF

(71) Applicant: ARIZONA BOARD OF REGENTS ON BEHALF OF ARIZONA STATE UNIVERSITY, Scottsdale, AZ (US)

(72) Inventors: Qiang CHEN, Chandler, AZ (US); Huafang LAI, Chandler, AZ (US)

(21) Appl. No.: 19/126,127

(22) PCT Filed: Nov. 1, 2023
(86) PCT No.: PCT/US2023/078319

§ 371 Date: Apr. 30, 2025

Related U.S. Application Data
(60) Provisional application No. 63/381,807, filed on Nov. 1, 2022.

(51) Int. Cl. C12N 15/82

(52) U.S. Cl. CPC C12N 15/8262; C12N 15/8203; C12N 15/8218; C12N 15/8257; C12N 2770/00023; C12N 2770/00042; C12N 2770/00051; C12N 2770/18023; C12N 2770/18042; C12N 2770/18051

(57) ABSTRACT

The present invention provides nucleic acid-loaded virus-like particles (VLPs) that can be produced in plants and are designed to deliver a nucleic acid into plant cells. Also provided are compositions comprising the nucleic acid-loaded VLPs, and methods of making and using the nucleic acid-loaded VLPs.

### CROSS-REFERENCE TO RELATED APPLICATIONS

This application claims the benefit of priority of U.S. Provisional Patent Application No. 63/381,807, filed Nov. 1, 2022. The contents of which is incorporated by reference herein in its entirety.

### SEQUENCE LISTING

A Sequence Listing accompanies this application and is submitted as an XML file of the sequence listing named “112624.01431_SL_ST26.xml” which is 61,805 bytes in size and was created on Oct. 30, 2023. The sequence listing is electronically submitted via Patent Center with the application and is incorporated herein by reference in its entirety.

### BACKGROUND

Genetic engineering of plants may play a critical role in environmental sustainability efforts, natural product synthesis for pharmaceuticals, and modification of agricultural crops to meet the needs of the growing human population in the changing global climate. Plant biotechnology is currently limited by the cost, difficultly, and throughput of methods for probing and modifying plant genetics. To improve such methods, new methods for delivering polynucleotides to target plant cells that are more efficient, safer, and environmentally friendly are needed.

### SUMMARY

In a first aspect, the present invention provides virus-like particles (VLPs) comprising a plant virus capsid protein and containing a replicon comprising a plant promoter operably linked to a heterologous polynucleotide. The 3′ end of the heterologous polynucleotide is flanked by a geminiviral short intergenic region (SIR) sequence, and the 5′ and 3′ ends of the replicon consist of a geminiviral LIR sequence.

In a second aspect, the present invention provides compositions comprising a VLP described herein and a carrier.

In a third aspect, the present invention provides methods for producing a nucleic acid-loaded VLP. The methods comprise (a) introducing into a plant cell a first construct comprising a first replicon that comprises a plant promoter operably linked to a first polynucleotide encoding a plant virus capsid protein, wherein the 3′ end of the first polynucleotide is flanked by a geminiviral SIR sequence, and wherein the 5′ and 3′ ends of the first replicon consist of a geminiviral LIR sequence; (b) introducing into the plant cell a second construct comprising a second replicon that comprises a plant promoter operably linked to a second, heterologous polynucleotide, wherein the 3′ end of the heterologous polynucleotide is flanked by a geminiviral SIR sequence, and wherein the 5′ and 3′ ends of the second replicon consist of a geminiviral LIR sequence; (c) introducing a third construct comprising a plant promoter operably linked to a third polynucleotide encoding Rep and RepA; and (d) harvesting the VLPs produced by the plant cell.

In a fourth aspect, the present invention provides methods of using a VLP described herein to deliver a heterologous polynucleotide to a plant cell. The methods comprise delivering the VLP to a plant.

### BRIEF DESCRIPTION OF THE DRAWINGS

FIG. 1 demonstrates that plant-made Norwalk virus capsid protein (NVCP) virus-like particles (VLPs) efficiently bind and enter mammalian cells. Various quantities of plant-made NVCP VLPs were incubated with Caco-2 cells. Then, the presence of VLPs on the surface of and inside Caco-2 cells was analyzed by staining the cells with anti-NVCP primary antibodies (1:500) and FITC-labelled secondary antibodies (1:200) and using flow cytometry to detect FITC florescence.

FIG. 2 demonstrates that smaller, deconstructed replicons result in more efficient protein expression in mammalian cells. A plant-based geminiviral replicon comprising a mammalian green fluorescent protein (GFP) expression cassette (i.e., pBY1050-GFP; ~6973 bp) was transfected into Caco-2 cells in parallel with a smaller, deconstructed geminiviral replicon comprising the mammalian GFP expression cassette (i.e., pBY-1193GFP; ~2619 bp). GFP expression was analyzed by microscopy.

FIG. 3 demonstrates that plants can be used to produce VLPs that are efficiently delivered into mammalian cells. NVCP VLPs loaded with replicons encoding GFP were produced in N. benthamiana plants, purified, and incubated with Caco-2 cells for 48 hours. GFP expression in the Caco-2 cells was then analyzed by quantitating florescence intensity using flow cytometry.

FIG. 4 demonstrates that Rep/RepA expression enhances GFP expression from the viral replicon. Caco-2 cells were transfected with a plasmid encoding the protein Rep/RepA (A). Caco-2 cells that were not transfected with the Rep/RepA plasmid were used as a negative control (B). 24 hours after transfection, equal amounts (3 μg) of plant-produced replicon-loaded NVCP VLPs were incubated with the Caco-2 cells. GFP expression was observed 48 hours after VLP incubation using a fluorescent microscope.

FIG. 5 demonstrates that GFP replicon copy number is increased in the presence of Rep/RepA. Caco-2 cells were transfected with a plasmid encoding the protein Rep/RepA (dots on right at each time point). Caco-2 cells that were not transfected with the Rep/RepA plasmid were used as a negative control (middle dots at each time point). 24 hours after transfection, equal amounts (3 μg) of plant-produced replicon-loaded NVCP VLPs were incubated with the Caco-2 cells. 48 hours after VLP incubation, quantitative PCR (qPCR) was performed using GFP-specific primers to analyze the copy number of GFP replicons in the Caco-2 cells. Caco-2 cells that were not incubated with VLPs were used as a negative control for PCR (left dots at each time point). Copy numbers are expressed as Ct values. (Note: A lower Ct value indicates that there is more DNA in sample.)

FIG. 6 demonstrates that genes delivered into mammalian cells by gene-encapsulating VLPs can result in the desired therapeutic effect. NVCP VLPs that encapsulated genes of GFP or A10, a protein with inhibitive activity against SARS-CoV-2 (e.g., a viral inhibitory agent), were produced in plants by co-infiltration of gene constructs of NVCP+GFP or NVCP+A10. Vero cells were incubated with NVCP VLPs and then infected with SARS-CoV-2. Cells were then fixed and stained for the spike protein of SARS-CoV-2 and plates were imaged on an ELISPOT reader for viral foci. The percentage of inhibition on viral infection was measured by foci reduction. *** indicates p value<0,0002.

FIG. 7A-7E provides maps of the vectors, referred to herein as (FIG. 7A) pBYR-1118NVCP (SEQ ID NO:1), (FIG. 7B) p1060mRep (SEQ ID NO:2), (FIG. 7C) p1232mRepA (SEQ ID NO:3), (FIG. 7D) pBY1050-GFP (SEQ ID NO:4), and (FIG. 7E) pBY-1193GFP (SEQ ID NO: 5).

### DETAILED DESCRIPTION

The present invention provides nucleic acid-loaded virus-like particles (VLPs) that are designed to deliver a nucleic acid into plant cells. Also provided are compositions comprising the nucleic acid-loaded VLPs, and methods of making and using the nucleic acid-loaded VLPs.

In the Examples, the inventors describe a novel platform in which a DNA replicon encoding a polypeptide (e.g., inhibitory protein or vaccine) is loaded inside a VLP during its assembly in planta. The polypeptide is placed under control of a constitutive promoter to allow for gene expression in a host cell after VLP uptake. This creates a potent delivery system that can be used to affect a variety of processes (e.g., viral infection; gene therapy, delivery of gene editing reagents) in vivo for the treatment of diseases and other conditions. While the Examples are directed to mammalian VLPs for delivery to mammalian cells similar plant VLPs for delivery to plant cells are made using similar procedures.

### Nucleic Acid-Loaded Virus-Like Particles:

In a first aspect, the present invention provides virus-like particles (VLPs) comprising a plant virus capsid protein, such as an icosahedral or rod-shaped virus capsid protein, and containing a replicon comprising a plant promoter operably linked to a heterologous polynucleotide. The 3′ end of the heterologous polynucleotide is flanked by a geminiviral short intergenic region (SIR) sequence, and the 5′ and 3′ ends of the replicon consist of a geminiviral LIR sequence.

As used herein, the term “virus-like particle (VLP)” refers to structures made of assembled viral proteins that are non-infectious because they lack some or all viral genetic content. VLPs can be derived from enveloped or non-enveloped viruses. VLPs can be naturally occurring or synthesized through the expression of viral structural proteins that can self-assemble into a virus-like structure. The VLPs of the present invention are “nucleic acid-loaded,” meaning that they contain nucleic acids that are encapsulated in the viral protein structure.

The term “capsid” refers to the protein shell of a virus, which encloses its genetic material. Viral capsids may consist of one or more proteins and are broadly classified according to their structure. The majority of viruses have capsids that have either a helical or an icosahedral structure. The VLPs may comprise an icosahedral or helical capsid protein. Icosahedral capsids, which may comprise 20 equilateral triangular faces, approximate a sphere and are made up of subunits that self-assemble into a VLP even in the absence of the viral genome. The icosahedral capsid protein used with the present invention maybe from a plant virus such that the VLPs can be used to deliver nucleic acids into plant cells. Several viral capsid proteins are amenable to insertions of heterologous sequences, which allows one to display a protein (e.g., a targeting moiety) on the surface of the VLP. The targeting moiety may allow a VLP comprising a mammalian capsid protein to target and invade a plant cell. The capsid protein used with the present invention is preferably from a plant virus such that the VLPs can be used to deliver nucleic acids into plant cells. Suitable plant virus capsid proteins for use in the VLPs include, without limitation, the cowpea mosaic virus (CPMV) coat proteins (i.e., large (L) and small(S) coat proteins), the cowpea chlorotic mottle virus (CCMV) coat protein, the brome mosaic virus (BMV) coat protein, the red clover necrotic mosaic virus (RCNMV) coat protein, the hibiscus chlorotic ringspot virus (HCRSV) coat protein, the tobacco mosaic virus (TMV) coat protein, and the potato virus X (PVX) coat protein. The capsid proteins listed above are from non-envelope plant viruses. To make VLPs based on enveloped plant viruses, such as sonchus yellow net virus (SYNV) and potato yellow dwarf virus (PYDV), the capsid protein can be replaced with the glycoprotein (G protein) and matrix protein of the virus.

A “replicon” is a nucleic acid molecule that replicates from a single origin of replication. The replicons used with the present invention include geminiviral genomic elements called long intergenic regions (LIRs), which allow them to replicate inside host cells in the presence of the proteins Rep and RepA via a rolling-circle mechanism that is described below. The VLPs of the present invention are designed to deliver a replicon comprising a heterologous polynucleotide into plant cells. This replicon is referred to herein as the “cargo replicon”.

As used herein, the term “promoter” refers to a DNA sequence that defines where transcription of a gene begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. It is understood by those skilled in the art that different promoters may direct the expression of a gene in different tissues or cell types, at different stages of development, or in response to different environmental conditions. Promoters that cause a gene to be expressed in most cell types at most times are commonly referred to as “constitutive promoters,” whereas promoters that allow for controlled expression of a gene (e.g., under particular conditions or in the presence of a particular molecule) are referred to as “inducible promoters.” Suitable promoters for use with the present invention include, but are not limited to, constitutive, inducible, temporally regulated, developmentally regulated, chemically regulated, tissue-preferred, and tissue-specific promoters. A promoter is “operably linked” to a gene if the promoter is positioned such that it can affect transcription of the gene.

The promoter included in the cargo replicon is a plant promoter. A “plant promoter” is a promoter that drives gene expression in plant cells. Commonly used plant promoters include, without limitation, the 35S promoter of the cauliflower mosaic virus, ubiquitin, tCUP cryptic constitutive promoter, the Rsyn7 promoter, the maize In2-2 promoter, and the tobacco PR-la promoter. Suitable plant promoters also include pathogen-inducible promoters, glucocorticoid-inducible promoters, alcohol-inducible promoters, estrogen-inducible promoters, and tetracycline-inducible/repressible promoters.

The terms “polynucleotide” and “nucleic acid” are used interchangeably to refer a polymer of DNA or RNA. A polynucleotide may be single-stranded or double-stranded and may represent the sense or the antisense strand. A polynucleotide may be synthesized or obtained from a natural source. The term polynucleotide encompasses constructs, plasmids, vectors, and the like.

The VLPs of the present invention contain a cargo replicon that includes a heterologous polynucleotide. As used herein, a “heterologous polynucleotide” is a polynucleotide that is not naturally found in the viral genome from which the VLP is derived.

In some embodiments, the heterologous polynucleotide included in the cargo replicon encodes a protein. The terms “protein,” “polypeptide,” and “peptide” are used interchangeably herein to refer to a polymer of amino acid residues connected to by peptide bonds between the alpha-amino and carboxy groups of adjacent residues. Proteins may include modified amino acids (e.g., phosphorylated, glycated, glycosylated, etc.) and amino acid analogs. The heterologous polynucleotide may encode any protein of interest. Suitable proteins include, without limitation, protein-based drugs (e.g., antibodies), antigens, gene editing reagents (e.g., Cas9), and homing proteins. Examples of protein-based drugs include the severe acute respiratory syndrome coronavirus 2 (SARS-CoV-2) inhibitory protein A10 (e.g., a viral inhibitory agent) (SEQ ID NO:8 and SEQ ID NO:9). Examples of antigen proteins include the spike(S) protein of SARS-CoV-2 and the envelope (E) protein of a flavivirus (e.g., West Nile virus, Dengue virus, Zika virus) an alpha virus (e.g., Chikungunya virus), a Filovirus (e.g., Ebola virus), a Lentivirus (e.g., human immunodeficiency virus (HIV)), or an influenza virus (e.g., influenza virus A).

As used herein, a “a viral inhibitory agent” refers to any protein or nucleic acid capable of inhibiting the life cycle of a virus, resulting is reduced viral load, reduced viral infection symptoms, or prevention of viral infection. Viral inhibitory agents may include but not be limited to antibodies that target viral proteins and nucleic acids that bind either viral proteins or viral genome sequences and RNAs.

As used herein, the term “homing protein” refers to a protein that interacts with a tissue or cell type-specific surface protein. Expression of a homing protein on the surface of a VLP targets the VLP to particular cells. Thus, in some embodiments, the VLP expresses the protein encoded by the heterologous polynucleotide on its surface.

In other embodiments, the heterologous polynucleotide encodes an RNA molecule. For example, the heterologous polynucleotide may encode a small interfering RNA (siRNA), short hairpin RNA (shRNA), anti-sense RNA, microRNA (miRNA), or guide RNA (gRNA).

The DNA replicon tested in the Examples is a deconstructed, single-stranded DNA plant virus replicon based on the bean yellow dwarf virus (BeYDV) of the Geminiviridae family. The BeYDV genome contains a long intergenic region (LIR) and short intergenic region (SIR) with four protein-encoding genes lying therebetween. The movement protein (MP) and the capsid protein (CP) genes occur on the sense (V) strand of the viral genome, while the replication initiator protein (Rep) and RepA protein genes lie on the complementary-sense (C) strand. Alternative splicing of the C transcript yields mRNAs for Rep and RepA. (Rep mRNA is generated when a short intron is spliced, while RepA mRNA is generated when this transcript is not spliced.) The LIR and SIR are the only cis-acting elements required for replication of the viral genome. The LIR contains a bi-directional promoter and a stem-loop structure that is essential for initiation of rolling-circle replication. The SIR is the origin of C-strand synthesis and contains transcription termination and polyadenylation signals. Geminiviral vectors are described in U.S. Pat. No. 10,125,373 and US Publication No. 20190336596, which are incorporated herein by reference.

Geminiviruses replicate DNA to high levels inside plant cells via a rolling-circle mechanism in which the Rep protein provides nicking and ligating functions. Initially, viral single-stranded DNA enters the cell nucleus and is converted into double-stranded DNA, which serves as the template for viral transcription and further replication. Rep nicks the positive sense (V) strand at a specific sequence within the LIR and then covalently binds to the 5′ terminus. The 3′-OH terminus is used as the primer for the synthesis of the nascent plus strand. DNA synthesis is accomplished by host replication proteins, including DNA polymerases. Completion of the nascent plus strand regenerates the origin of replication, which is nicked by the Rep protein again. The Rep protein then acts as a terminase to release the displaced plus strand and simultaneously ligates it into a circular form. In the process, Rep is transferred to the newly created 5′ terminus. Late in the replication cycle single-stranded viral genomes are generated for encapsidation. RepA has an autoregulatory effect on Rep gene expression. It binds to a Rep binding site located within the LIR and is hypothesized to play a role in inhibiting complementary-sense gene expression and trans-activating sense gene expression. RepA has also been observed to bind the retinoblastoma-related protein (RBR), suggesting that it may help to arrest the infected plant cell in S-phase by interacting with RBR, thereby, creating conditions favorable for viral DNA replication. In plants, both Rep and RepA are required for replicons to self-replicate and the ratio of these proteins affects replication efficiency.

To allow the replicons used with the present invention to be replicated in plant and/or mammalian cells, the replicons comprise geminiviral long intergenic region (LIR) sequences at their 5′ and 3′ ends. As used herein, “a geminiviral long intergenic region (LIR)” is a portion of a geminiviral genome that contains a Rep binding site that is capable of mediating excision and replication by a geminiviral Rep protein. Thus, the LIRs define the portion of a construct that will be replicated as part of the replicon via the rolling-circle mechanism described above. An exemplary geminiviral LIR sequence from bean yellow dwarf virus (BeYDV) is provided as SEQ ID NO:6.

The replicons may also comprise a geminiviral short intergenic region (SIR) sequence. As used herein, “a geminiviral short intergenic region (SIR)” is a portion of a geminiviral genome that contains bidirectional transcription terminator signals. SIRs are suspected to be the origin of complementary strand synthesis. In the cargo replicon, SIR flanks the heterologous polynucleotide on its 3′ end to aid in transcriptional termination. An exemplary geminiviral SIR sequence from bean yellow dwarf virus (BeYDV) is provided as SEQ ID NO:7.

The replicons described herein can be made to be self-replicating by inclusion of a polynucleotide encoding Rep and RepA in the replicon. Alternatively, Rep and RepA can be provided in trans (i.e., on a separate construct) from the heterologous polynucleotide. In the BeYDV genome, Rep and RepA are generated from the same transcript via alternative splicing. Thus, in some embodiments, the VLPs comprise a polynucleotide encoding both Rep and RepA. In other embodiments, Rep and RepA are encoded by two separate polynucleotides. In some embodiments, the polynucleotide(s) encoding Rep and/or RepA are operably linked to an inducible promoter such that replicon replication can be controlled.

As used herein, the term “construct” refers to a recombinant polynucleotide. A construct may be single-stranded or double-stranded and may represent the sense or the antisense strand. A “recombinant polynucleotide” is an artificially constructed polynucleotide that includes polynucleotide sequences derived from at least two different sources. The constructs described herein may be prepared using standard techniques such as cloning, DNA and RNA isolation, amplification, and purification.

Compositions for Delivery into Plant Cells:

In a second aspect, the present invention provides compositions comprising a VLP described herein and a carrier for delivery into plant cells.

Many suitable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, and suspensions, including saline and buffered media.

The compositions of the present invention may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances, or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

The compositions may be packaged into vials or other suitable containers, including individual or multi-dose ampoules.

The compositions may be lyophilized before packaging, allowing them to be stored for extended periods of time without loss of viability at ambient temperatures. The term “lyophilization,” as used herein, refers to freezing of a material at low temperature followed by dehydration by sublimation, usually under a high vacuum. Lyophilization is also known as freeze drying. Many freezing techniques can be used in lyophilization, such as tray-freezing, shelf-freezing, spray-freezing, shell-freezing, and liquid nitrogen immersion. Each technique will result in a different rate of freezing.

### Methods for Making Nucleic Acid-Loaded Virus-Like Particles:

In a third aspect, the present invention provides methods for producing a nucleic acid-loaded VLP. The methods comprise (a) introducing into a plant cell a first construct comprising a first replicon that comprises a plant promoter operably linked to a first polynucleotide encoding a plant virus capsid protein, wherein the 3′ end of the first polynucleotide is flanked by a geminiviral SIR sequence, and wherein the 5′ and 3′ ends of the first replicon consist of a geminiviral LIR sequence; (b) introducing into the plant cell a second construct comprising a second replicon that comprises a plant promoter operably linked to a second, heterologous polynucleotide, wherein the 3′ end of the heterologous polynucleotide is flanked by a geminiviral SIR sequence, and wherein the 5′ and 3′ ends of the second replicon consist of a geminiviral LIR sequence; (c) introducing a third construct comprising a plant promoter operably linked to a third polynucleotide encoding Rep and RepA; and (d) harvesting the VLPs produced by the plant cell.

The present methods involve introducing at least three constructs into a plant cell. The first construct includes a first replicon that drives the expression of a plant virus capsid protein in plant cells. This replicon is used to produce the VLP capsid in the plant cell and is, thus, referred to herein as the “packaging replicon”. The second construct includes a second replicon that drives the expression of a gene product (i.e., an RNA or protein) encoded by the heterologous polynucleotide in plant cell. This replicon is the cargo that is packaged into VLPs for delivery to a target plant cell and is, thus, referred to herein as the “cargo replicon.” Notably, during VLP production, the cargo replicon will be preferentially included in the VLPs due to its small size relative to the packaging replicon. The third construct drives the expression of Rep and RepA in plant cells, which allows the first and second replicons (which both include LIR sequences at their 5′ and 3′ ends) to be replicated in the plant cell. In some embodiments, the third construct comprises an inducible plant promoter such that Rep/RepA expression, and therefore replicon replication, can be controlled. In some embodiments, Rep and RepA are expressed from two different constructs (i.e., the third construct and an additional, fourth construct).

These constructs may be introduced into the plant cell using any known method of plant cell transfection including, without limitation, Agrobacterium-mediated transformation and particle bombardment. In some embodiments, one or more of the first construct, the second construct, and the third construct are integrated into the genome of the plant cell. In other embodiments, the transfection is transient.

In some embodiments, the plant cell is part of a plant. The term “plant” includes plant protoplasts, plant cell tissue cultures from which plants can be regenerated, plant calli, plant clumps, and plant cells that are intact in plants, as well as plant parts, such as embryos, pollen, ovules, flowers, glumes, panicles, leaves, stems, roots, root tips, anthers, and pistils. In some embodiments, the transfected plant cell is part of a leaf of the plant.

The plant cell used to produce the VLP may be from any species of plant that supports VLP production and replicon replication, including both dicotyledonous and monocotyledonous plants. Suitable plants dicotyledonous plants include, without limitation, tobacco, potato, Nicotiana benthamiana, tomato, cotton, beans, spinach, alfalfa, and lettuce plants. Monocotyledonous plants include, without limitation, rice, maize (corn), wheat, barley, oat, rye, sorghum, and millet. Nicotiana benthamiana is a relative of tobacco that is a useful host for recombinant protein expression from viral vectors because it supports the replication of many different viruses. Thus, in some embodiments, the plant is Nicotiana benthamiana.

Expression of both the capsid protein (from the first construct) and the Rep/RepA proteins (from the third construct) in the plant cell is driven by a plant promoter. A “plant promoter” a promoter that drives gene expression in plant cells. Suitable plant promoters for use in these constructs include, without limitation, the 35S promoter of the cauliflower mosaic virus promoter, ubiquitin promoter, tCUP cryptic constitutive promoter, Rsyn7 promoter, maize In2-2 promoter, and tobacco PR-la promoter. Suitable plant promoters also include pathogen-inducible promoters, glucocorticoid-inducible promoters, alcohol-inducible promoters, estrogen-inducible promoters, and tetracycline-inducible/repressible promoters.

The VLPs produced by the plant cell may be harvested using any suitable method known in the art. In some embodiments, the VLPs are purified away from the plant material using centrifugation-based methods such as rate-zonal density, isopycnic, and gradient density centrifugation; or chromatography-based methods such as ion-exchange, affinity, hydrophobic interaction, or metal-chelation chromatography. VLPs can also be purified by various filtration method such as diafiltration and ultrafiltration, and by precipitation method such as low pH precipitation. In some embodiments, the VLPs are harvested at least 3 days after the constructs are introduced into the plant cell.

After the VLPs have been harvested, they can be used to introduce the cargo replicon into a target plant cell. The VLPs can be used to introduce nucleic acids into both cultured plant cells (i.e., by adding the VLPs to the culture) and plant cells in vivo (i.e., by delivering the VLPs to a plant and allowing them to infect any cells that are bound by the viral capsid protein).

Any of the constructs used in these methods (i.e., the first construct, the second construct, the third construct, and/or the fourth construct) may be included together on one polynucleotide. Specifically, two of the constructs, three of the constructs, or all four of the constructs may be provided as a single polynucleotide. In embodiments in which the first replicon and the second replicon are provided as a single polynucleotide, these replicons can be linked in tandem such that both replicons share a geminivirus LIR positioned between the replicons. In some embodiments, the third construct is included on the same polynucleotide as the first construct or the second construct. However, in preferred embodiments, the third construct is included on the same polynucleotide as the first construct such that the second construct remains small and is preferentially incorporated into VLPs during assembly in plant cells.

### Methods for Using Nucleic Acid-Loaded Virus-Like Particles:

In a fourth aspect, the present invention provides methods of using a VLP described herein to deliver a heterologous polynucleotide to a plant cell. The methods comprise delivering the VLP to a plant.

In some embodiments, the methods are used to modulate the expression of a gene in the plant. In these embodiments, the heterologous polynucleotide encodes a gene product that modulates gene expression. For example, the heterologous polynucleotide may encode a microRNA (miRNA) or a small interfering RNA (siRNA) that is used to repress the expression of a gene via RNA interference. RNA interference (RNAi) is a natural mechanism for sequence-specific gene silencing. In RNAi, miRNAs or siRNAs can either (1) direct enzyme complexes to degrade messenger RNA (mRNA) molecules and thus prevent their translation, or (2) inhibit transcription of the mRNA molecules by directing an enzyme complex to catalyze DNA methylation at specific genomic positions.

Alternatively, the heterologous polynucleotide may encode a gene editing reagent that is used to edit a gene. Gene editing may involve the introduction of deletions or insertions into a native gene, integrations of exogenous DNA, gene correction, and/or gene mutation. Gene editing can be used to introduce a heterologous polynucleotide into the genome; to silence, reduce, or increase the expression of a native gene; or to modify the gene product produced by a native gene.

In some embodiments, the gene editing reagent is a zinc-finger nuclease, TALEN, or nucleic-acid guided nuclease. As used herein, a “nucleic acid-guided nuclease” is any nuclease that cleaves DNA, RNA or DNA/RNA hybrids, and which uses one or more guide nucleic acids (gNAs) to confer specificity. A nucleic acid-guided nuclease can be a DNA-guided DNA nuclease, a DNA-guided RNA nuclease, an RNA-guided DNA nuclease, or an RNA-guided RNA nuclease. A nucleic acid-guided nuclease can be an endonuclease or an exonuclease. A nucleic acid-guided nuclease may be naturally occurring or engineered. In some embodiments, the nucleic acid-guided nuclease is selected from the group consisting of Cas9, Cpf1, Cas3, Cas8a-c, Cas10, Cas13, Cas14, Cse1, Csy1, Csn2, Cas4, Csm2, Cm5, Csf1, C2c2, CasX, CasY, Cas14, and NgAgo. The nucleic acid-guided nuclease can be from any bacterial or archaeal species. For example, in some embodiments, the nucleic acid-guided nuclease is from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis Corynebacter diphtheria, Acidaminococcus, Lachnospiraceae bacterium, or Prevotella.

In some embodiments, the gene editing reagent is a guide nucleic acid. A “guide nucleic acid (gNA)” is a nucleic acid that targets a nucleic acid-guided nuclease to a specific genomic sequence (i.e., the target sequence) via complementary base pairing. The complementary portion of a gNA comprises at least 10 contiguous nucleotides, and often comprises 17-23 contiguous nucleotides that are complementary to the target sequence. The complementary portion of the gNA may be partially or wholly complementary to the target sequence. In some embodiments, the gNA is from 20 to 120 bases in length, or more. In certain embodiments, the gNA can be from 20 to 60 bases, 20 to 50 bases, 30 to 50 bases, or 39 to 46 bases in length. Various online tools and software environments can be used to design an appropriate gNA for a particular application. The gNA may comprise DNA and/or RNA. In some embodiments, the gNA is a chemically modified gNA. For example, the gNA may be chemically modified to decrease a cell's ability to degrade the gNA. Suitable gNA chemical modifications include 2′-fluoro (2′-F), 2′-O-methyl(2′-O-Me), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the gNA is composed of two molecules that base pair to form a functional gRNA: one comprising the region that binds to the nucleic acid-guided nuclease and one comprising a targeting sequence that binds to the target sequence. Alternatively, the gNA may be a single molecule comprising both of these components, e.g., a single guide RNA (sgRNA).

As used herein, the term “delivering” refers to the introduction of a substance into a plant. Suitable routes for delivering the VLPs described herein include cutting the plant, wounding the plant, vector-based transmission (e.g., aphid transmission), chemical treatment, vacuum infiltration, and particle bombardment.

The plant cell to which the heterologous polynucleotide is delivered may be from any species of plant and any part of a plant so long as the plant virus capsid protein can bind to the cell. The target plant cell may be a cultured plant cell or a plant cell that is part of a plant or a plant part.

The methods of the present invention may be used to improve or introduce a trait of agronomic interest within the plant. Examples of such traits include, without limitation, herbicide resistance, resistance against a bacterial, fungal, or viral infection, insect resistance, pest resistance, resistance to extreme temperatures, drought resistance, male sterility, male fertility, enhanced nutritional quality, industrial usage, yield stability, higher seed yield, and yield enhancement. Many examples of genes that confer such traits have been described in the literature and are well known in the art.

In some embodiments, the plant is a crop. A “crop” is a plant that can be grown and harvested for profit or subsistence. In these embodiments, the methods may be used to increase the yield of the crop or improves the taste of the edible portion of the crop.

In some embodiments, the plant produces a protein or gene product that may be a therapeutic such as an antibody. The plant can be used to administer the therapeutic protein to a subject. The subject may have a disease, and the heterologous polynucleotide encodes a gene product that treats the disease. Any disease that can be treated using a protein-based drug, RNA-based drug, or gene editing may be treated using the methods of the present invention.

As used herein, “treating” or “treatment” describes the management and care of a subject for the purpose of combating a disease, condition, or disorder. Treating includes means to prevent the onset of the symptoms or complications, to alleviate the symptoms or complications, or to eliminate the disease, condition, or disorder.

In some embodiments, the plants generated herein are used to induce (e.g., cause) a therapeutic effect in the subject that is suffering from a disease, debilitating condition or injury. Diseases may include any infectious disease, deficiency disease, or immune disease. For example, the disease may include a viral disease, such as influenza and SARS-CoV-2. To induce a therapeutic effect of a viral disease, the VLPs may include a heterologous polynucleotide that encodes a viral inhibitory polypeptide. In these embodiments, the VLP may comprise an antibody, protein binding fragment, or antigen binding fragment that binds a viral component, causing inhibition of the virus. In some embodiments, the heterologous polynucleotide encodes A10, a polypeptide known to have inhibitory activity against SARS-CoV-2.

As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments (e.g., antigen binding fragments) as well as variants (including derivatives) of antibodies, antibody multimers and antibody fragments. Examples of molecules which are described by the term “antibody” herein include: single chain Fvs (scFvs), Fab fragments, Fab′ fragments, F(ab′)2, disulfide linked Fvs (sdFvs), Fvs, and fragments comprising or alternatively consisting of, either a VL or a VH domain.

As used herein, the term “antigen” refers to a molecule that can initiate a humoral and/or a cellular immune response in a recipient. Common categories of antigens include, but are not limited to, viral antigens, bacterial antigens, fungal antigens, protozoa and other parasitic antigens, tumor antigens, and antigens involved in autoimmune disease, allergy, and graft rejection. In some embodiments, the antigen is a disease-specific antigen, i.e., an antigen associated with or specific to a disease. In some embodiments, the antigen is a tumor antigen, i.e., an antigen that is preferentially expressed on the surface of a tumor cell and not expressed on normal, healthy cells.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

In the following example, the inventors describe a novel method for delivering a heterologous polynucleotide into a target cell. In their method, a DNA replicon comprising a heterologous polynucleotide is loaded into plant-based virus-like particles (VLPs) during assembly in planta in Nicotiana benthamiana. The heterologous polynucleotide is placed under the control of a constitutive mammalian promoter to allow it to be expressed in the cells of an inoculated mammalian host following VLP uptake. A similar system is under development for delivery to plants using a constitutive plant promoter to drive expression of a heterologous polynucleotide for expression in a plant without risk of infection by a plant virus.

The DNA replicon that was tested by the inventors is a deconstructed, single-stranded DNA plant viral replicon from the bean yellow dwarf virus of the Geminiviridae family. This plant virus replicates to high levels inside plant cells via a rolling circle mechanism. Its replication is dependent on two short gene sequences, i.e., the long intergenic region (LIR) and the short intergenic region (SIR), as well as at least one of the replication associated proteins, Rep and RepA. The inventors demonstrate that this replicon is self-replicating in both plant and mammalian cells if all these genomic elements are included in it.

### Results

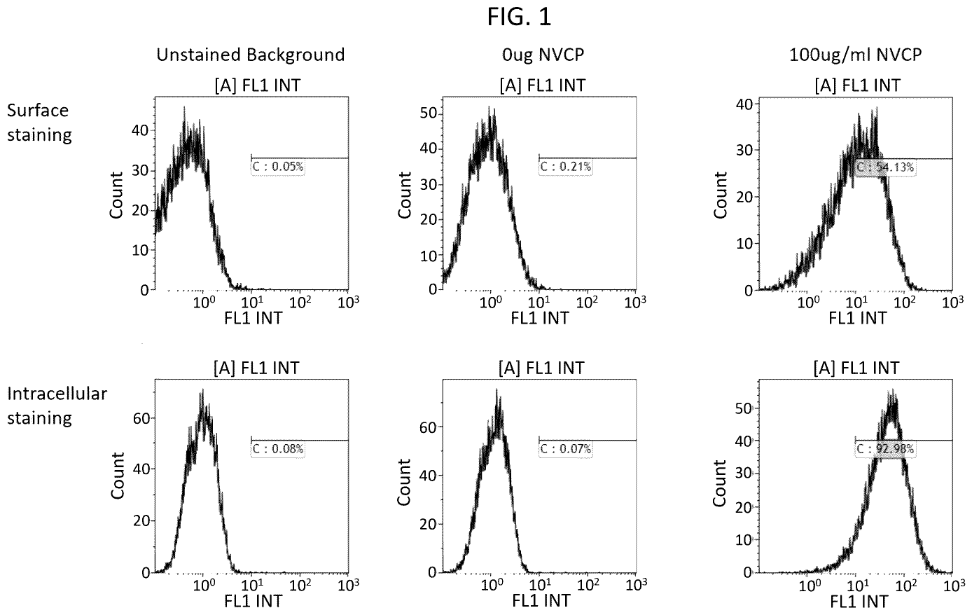

For proof-of-concept of replicon encapsulation by VLPs in planta as well as delivery, gene-of-interest expression, and replicon replication via Rep/RepA in mammalian cells, we utilized the Norwalk virus capsid protein (NVCP) VLP as a model VLP in conjunction with Caco-2 cells. We verified that plant-made NVCP VLPs bind to and enter Caco-2 epithelial cells efficiently (FIG. 1), validating this as a suitable system for assessing the feasibility of our DNA-loaded VLPs.

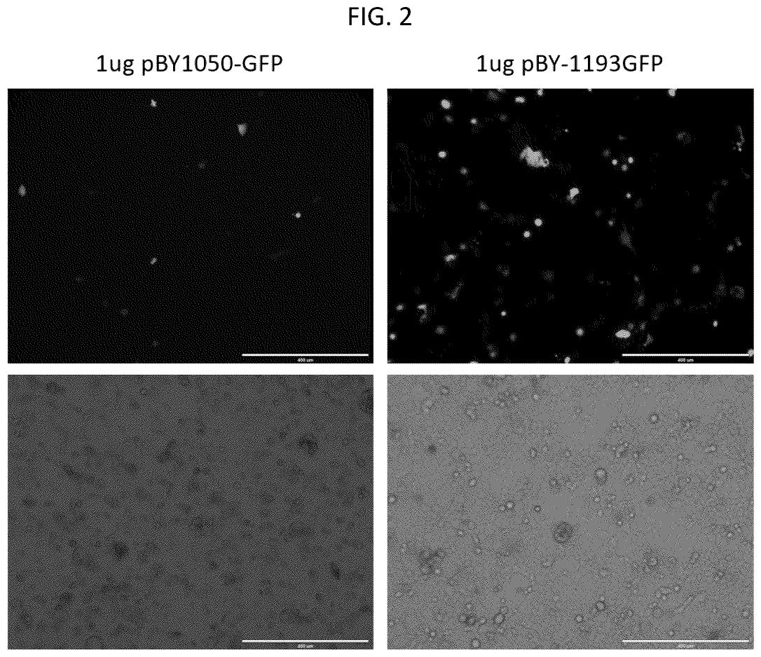

To assess the effect of replicon size on heterologous protein expression, the plant-based expression vector used to produce the VLPs (i.e., a vector containing the same LIR, SIR, and Rep/RepA genomic elements described above) was transfected into Caco-2 cells in parallel with a deconstructed replicon comprising a sequence encoding green fluorescent protein (GFP) under control of a constitutive mammalian promoter. As expected, the smaller, deconstructed replicon (pBY-1193GFP, SEQ ID NO:5) was much more efficient at expressing GFP than the larger plant replicon (pBY1050-GFP, SEQ ID NO:4) when equal masses of DNA were transfected (FIG. 2).

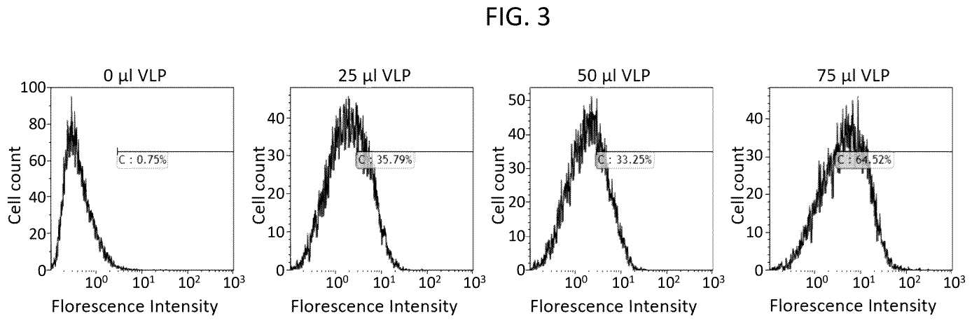

The ability of plants to produce NVCP VLPs that encapsulate the GFP replicons was tested by simultaneously introducing the NVCP and GFP replicons into N. benthamiana. VLPs were isolated from these plants and were incubated with Caco-2 cells. GFP expression was observed in a significant percentage of Caco-2 cells (FIG. 3), as measured by flow cytometry.

Overall, incubating the cells with increasing amounts of replicon-loaded VLPs resulted in increased GFP expression. These results demonstrate that (1) plants can be used to produce VLPs that carry a cargo replicon that can be efficiently delivered into mammalian cell, and (2) that the cargo replicon can direct the synthesis of a desired protein inside the infected mammalian cells.

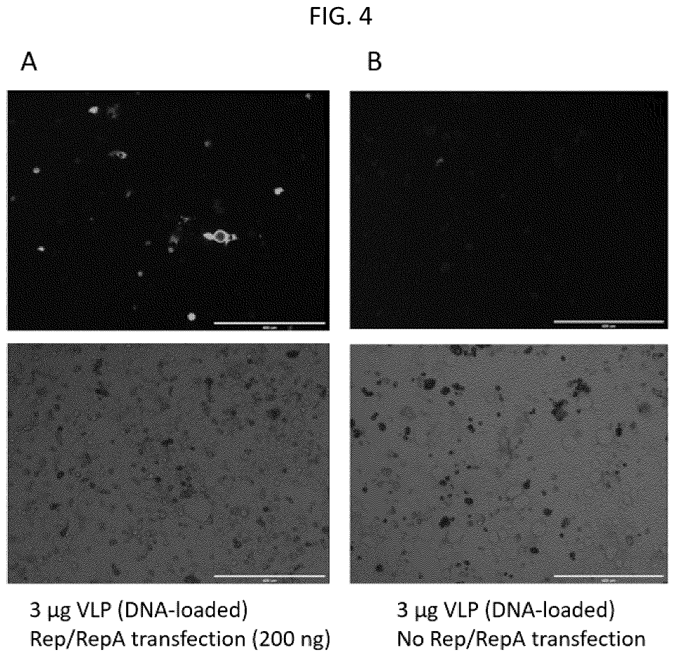

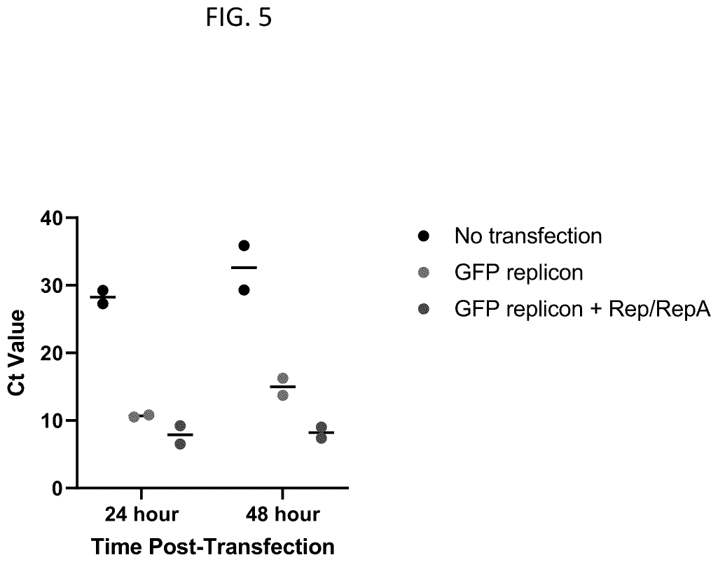

Enhanced GFP expression was observed in Caco-2 cells using a fluorescent microscope when vectors encoding Rep/RepA (p1060mRep, SEQ ID NO:2; p1232mRepA, SEQ ID NO:3) were transfected into the cells 24 hours before the DNA-loaded NVCP VLPs were introduced (FIG. 4). These data demonstrate that the replicons delivered by plant-produced VLPs replicate in mammalian cells in a Rep/RepA dependent manner. qPCR analysis of transient transfection of separate replicons encoding GFP and Rep/RepA showed that there was an increase in the GFP replicon copy number in the presence of Rep/RepA as compared to in its absence (FIG. 5), confirming the results obtained using fluorescent microscopy.

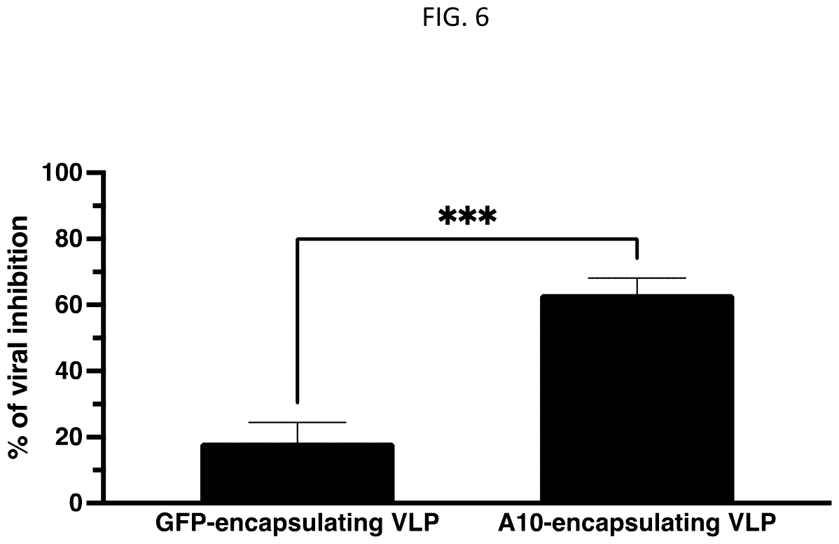

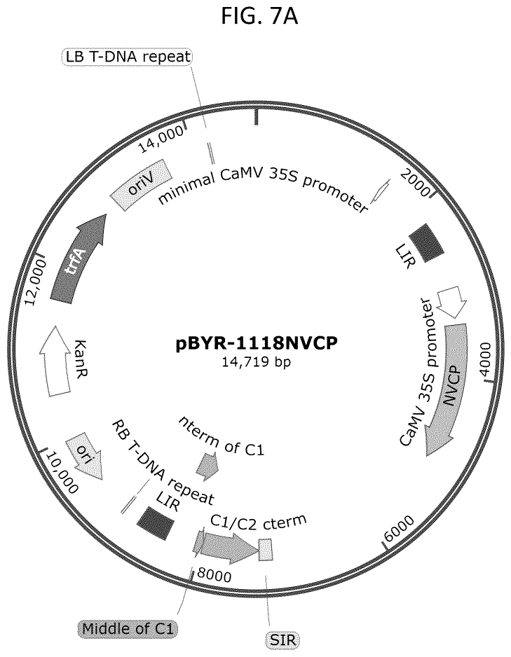

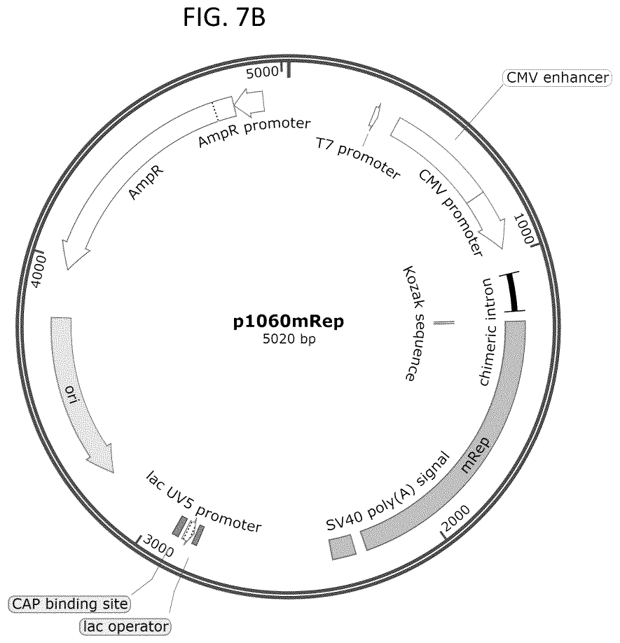

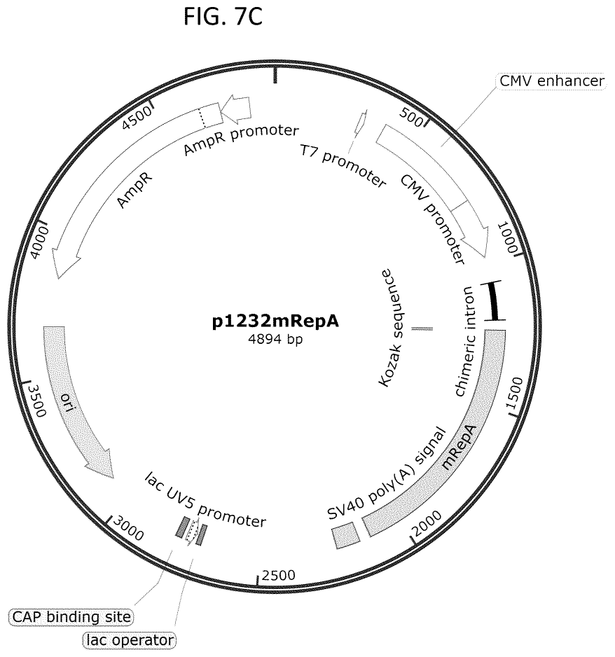

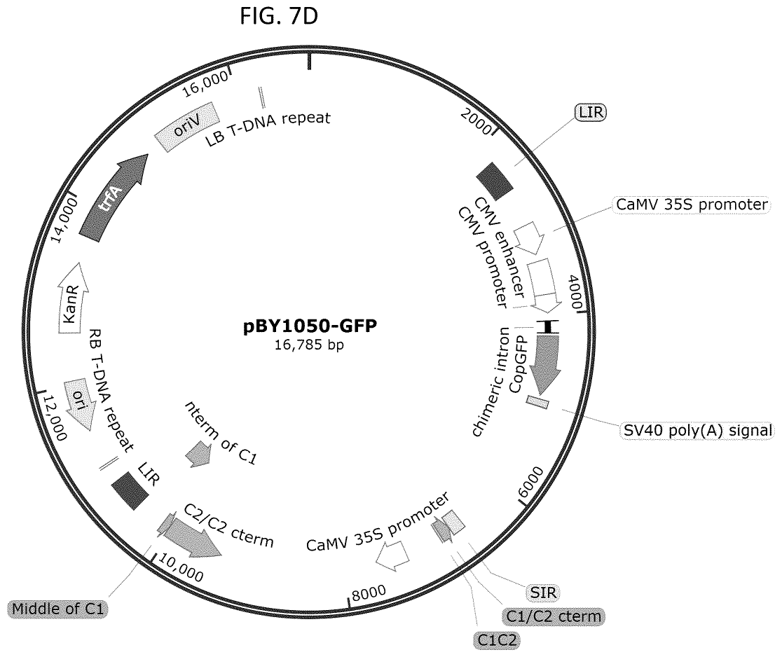

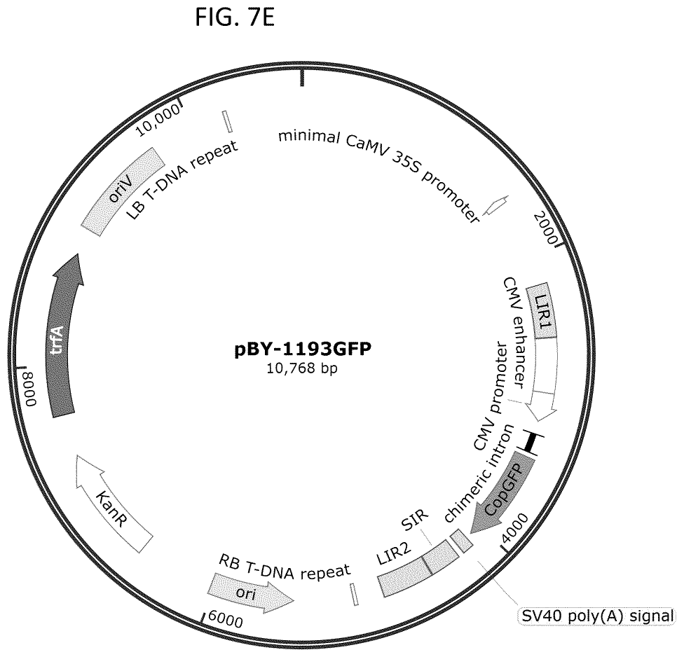

Vero cells were also shown to be successfully transduced by the VLPs (FIG. 6).

Furthermore, Vero cells transduced with VLPs containing a heterologous sequence encoding the SARS-CoV-2 viral inhibitory agent A10 showed increased viral inhibition over Vero cells transduced with VLPs containing a heterologous sequence encoding GFP when subsequently infected with SARS-CoV-2. These results suggest that the VLPs are capable of transducing mammalian cells and expressing in the mammalian cell a functional protein (e.g., encoded by the heterologous sequence) that is capable of inhibiting viral challenge. These results also suggest that the VLPs may be suitable for treatment of disease in a subject, such as for treatment or prevention of viral infections.

Taken altogether, these data suggest that a self-replicating VLP vaccine or therapeutic pharmaceutical is achievable with this technology.

### Discussion

Since RNA replicons derived from deconstructed plant viruses are routinely used in our laboratory, our self-replicating, dual VLP-DNA vaccine platform can be easily adapted to use plants to produce VLPs that encapsulate RNA replicons. Therefore, we refer to this technology platform as a VLP-nucleic acid (NA) dual system.

Our self-replicating, dual VLP-NA system is versatile. Many diverse and multivalent vaccines, therapeutic polypeptides, and nucleic acids can be generated from this single VLP. For example, one could design the VLP to display a heterodimer of an antigen (that is known to form dimers) from two different viruses (e.g., ZIKV and WNV), while incorporating the antigen gene from a different virus (e.g., one of the DENV serotypes) as the DNA component to make a trivalent vaccine. This type of multivalent vaccine would offer protection against multiple viruses or viral serotypes, and/or subvert safety concerns of antibody dependent enhancement of infection (ADE) in the case of DENV infection. Thus, it is possible to mix and match vaccine targets for different diseases that circulate together, particularly for globally spread viruses of concern, such as influenza and SARS-CoV-2. This strategy is beneficial in that fewer doses would be needed to inoculate against multiple viruses, which would reduce the cost of production and simplify the logistics involved in vaccine delivery to at-risk populations.

The VLP-NA system could also be used for other applications, such as gene therapy delivery. For example, it can be used to repress gene expression in a mammalian host through RNA interference (RNAi). This can be accomplished via DNA-based RNAi or direct encapsulation of the RNA construct during VLP assembly and production. Using the single-stranded DNA replicon system described herein, a sequence that will form a short hairpin RNA (shRNA) upon transcription can be introduced into a mammalian cell such that it is processed by host cellular machinery to generate a short interfering RNA (siRNA) that degrades a host mRNA to repress a gene of interest. If prolonged delivery of an siRNA is needed to control or prevent a disease state, the VLP-NA can be designed to be self-replicating.

Encapsulation of RNA during VLP production using this system also has broad ranging potential for gene therapy and vaccine development. A deconstructed RNA virus encapsulated in the VLP can be utilized as a gene expression tool, so long as elements of the rationally designed mRNA construct interact with the appropriate host factors and drive expression of the gene of interest. This would make the system comparable to the recently popularized Pfizer and Moderna mRNA vaccines against COVID-19.

Another useful aspect of this system outside vaccinology is that the VLP can be optimized to target specific tissue and cell types for delivery of the nucleic acid payload, which is important for gene therapy. Like the well-known adeno-associated viruses (AAVs), a VLP can have an inherent avidity for a cell or tissue type, or it can be genetically engineered to display a peptide or protein ligand to interact with a cell type-specific receptor. Fusing part (or all) of a specific AAV capsid for surface display in this system would allow for specific tropism to certain cell types and would be more favorable for regulatory authorities, in that AAVs have been well studied and a few AAV-based gene therapies have been approved by the FDA.

The usefulness of the system described here goes beyond the potential of the AAV system. Use of AAVs are subject to host pre-exposure to the AAV being used and since most people are exposed to many serotypes of AAVs throughout the natural course of life, immune responses against them may limit the effectiveness of the gene therapy. Although the virus itself is not known to cause disease, there is potential for AAVs to integrate into the host genome, albeit at lower frequency with recombinant genomes that are used for gene therapy. Although antibodies have been discovered in humans against a plant virus, it is widely accepted that plant viruses are not capable of causing disease in mammalian cells, do not integrate into human genomes and neither of these events have been observed in nature. The self-replicating nature of our deconstructed plant virus-based replicon ensures a prolonged delivery of the gene of interest, as well as increased safety over AAVs by utilizing plant viral elements for gene therapy applications. Since we can display the desired ligand protein on the surface of VLPs on demand, our technology can also allow the specific delivery of the NA cargo to the target cells, thereby, (1) minimizing the off-target side effect, another impediment of gene therapy, and (2) eliminating any safety concerns associated with the use of AAV or any other animal viruses.

This system can also be used for delivery of protein-based drugs via the packaged nucleic acid. Intracellular delivery of nucleic acid that encodes an antibody, antibody derivative, or antigen binding fragment is possible and many diseases would benefit from specific intracellular targeting and suppression of protein-protein interactions. The high specificity and affinity for the target epitope offers “intrabodies” a unique option for therapeutic treatments. Although the idea of intracellular antibody delivery has been around for decades, many technical hurdles, particularly an efficient delivery method of the intrabody (or DNA/RNA encoding it), have limited the potential of intrabody therapies. The VLP-NA system, along with other advances in intrabody development, can overcome these limitations and open many novel avenues for disease treatment. Through its self-replicating nature, this system has potential to deliver a targeted, substantial dose of the antibody drug in nucleic acid format to have a significant therapeutic effect on the disease state by interfering directly or indirectly with the pathogen. Diseases that have potential to benefit from this include many different cancers, viral diseases (after viral entry to the host cell), tuberculosis, and malaria, among others.

This system can also be used to deliver nucleic acid-based tools into a target organism or cells to alter specific gene expression for multiple applications. For example, the VLPs can deliver CRISPR/cas9 in the format of DNA or RNA to target cells for gene editing. This will have multiple applications in treating various diseases including genetic disorders. As discussed above, our system will (1) overcome the difficulty in delivering CRISPR/cas9 into cells, (2) make the delivery more specific to target cells by surface display of a specific ligand on the surface of VLP, reducing the off-target effect, and (3) address the safety concern of using animal virus vectors. In addition to medical applications, this technology can also be used for gene editing for scientific research purposes or to improve the traits of an organism. For example, it can be used to improve a plant's resistance to drought, extreme temperatures, insects/pests, or infections by viruses, bacteria, or fungi. It also can be used to increase crop yield or make crops resistant to herbicides.

Overall, this system, in which a deconstructed plant virus replicon is allowed to undergo a limited period of replication inside mammalian cells, has far-reaching implications for disease prevention and treatment. The encapsulation of DNA or RNA inside a recombinant VLP has only been reported in vitro after VLP purification. To our knowledge, we are the first to describe a functional mammalian expression cassette engineered into a plant virus replicon and to encapsulate the replicon inside a VLP during production in vivo. Furthermore, due to the inherent replication mechanism of the plant virus the replicon is based upon, encapsulation of the Rep/RepA gene on a separate replicon alongside a gene of interest in a single VLP allows for at least one round of replication of the replicon inside the target cell, enhancing the potency of the delivered nucleic acid. This method for nucleic acid encapsulation and cellular delivery has many applications including vaccine development, gene therapy, RNAi, and gene editing, among others, and has potential to facilitate safer and more efficacious treatments for a wide range of diseases.

### Materials & Methods

Design and Production of Virus-Like Particles (VLPs) in Nicotiana benthamiana

The capsid protein gene of Norwalk virus was modified from to include restriction enzyme recognition sites and was subcloned into a geminiviral vector (pBYR2eAK2Mc) based on the bean yellow dwarf virus for plant expression. Briefly, transformed EHA105 Agrobacterium tumefaciens containing the plant codon optimized NVCP construct (pBYR-1118NVCP, SEQ ID NO:1) or the deconstructed viral vector containing the mammalian GFP construct (pBY-1193GFP, SEQ ID NO:5) were grown in YenB media supplemented with 100 μg/mL of kanamycin and 0.5 μg/mL of rifampicin. After overnight incubation with shaking at 30 °C, cells were pelleted for 10 minutes at 6000×g and resuspended in MES buffer, pH 5.5. A 1:1 ratio (as measured by OD₆₀₀) of the pBYR-1118NVCP and pBY-1193GFP-containing A. tumefaciens strains (in combination, these two strains produce VLPs encapsulating a nucleic acid encoding GFP) or the pBYR-1118NVCP strain alone (which produces VLPs that lack nucleic acid cargo) were syringe infiltrated into N. benthamiana leaves as previously described. After 3 days, plant leaves were harvested and the NVCP-VLP alone or the NVCP-VLP harboring the pBY-1193GFP replicon were purified by sucrose gradient centrifugation as previously described. Purified NVCP-VLPs were then characterized as described above.

### Transient Transfection of Caco-2 Cells

Caco-2 cells were plated at 10,000 cells/well in 100 μl DMEM complete medium in 96-well tissue culture plate the day before transfection. Lipofectamine™ 3000 (Thermofisher) was used for transient transfection of plasmids encoding GFP, Rep, or RepA, according to the manufacturer's instructions. Typically, 0.1 μg of plasmid DNA was diluted in 5 μl Opti-MEM (Thermofisher) with 0.2 μl of P3000 reagent and then mixed with 0.2 μl of Lipofectamine™ 3000 diluted in 5 μl Opti-MEM. After a 10-minute incubation at room temperature, 10 μl of the DNA/Lipofectamine™ 3000 mixture was added to each well of Caco-2 cells. GFP expression was visualized, and images were taken 24, 48, or 72 hours after transfection using the Evos Imaging System (Invitrogen).

### Norwalk Virus Capsid Protein (NVCP) Staining

Caco-2 cells were plated at 0.5 million/well in 6 well plates the day before the experiment. The next day, the media in each well was replaced with 1 ml fresh complete culture media and then 100 μg/ml of Norwalk virus capsid protein (NVCP) in PBS was added to experimental wells, and an equal volume of PBS was added to negative control wells (0 μg NVCP). The cells were incubated with NVCP for 1 hour at 37 °C. After the incubation, the cells were washed twice with PBS and then scraped from the wells using a cell scraper (VWR) and resuspended in PBS on ice.

For cell surface staining, each sample was incubated with guinea pig anti-NVCP serum (1:500) for 1 hour and then washed twice with PBS and incubated with FITC goat anti-guinea pig (1:200) for 30 minutes. Finally, the samples were washed twice with PBS and resuspended in 400 μl PBS.

For intracellular staining, the samples were fixed with 4% paraformaldehyde (Electro Microscopy Sciences) in PBS at room temperature for 10 minutes and were then washed once with PBS and permeabilized with 0.1% saponin (Sigma) for 15 minutes. After that, samples were stained with guinea pig anti-NVCP serum and FITC goat anti-guinea pig as described above. Both NVCP on the Caco-2 cell surface and intracellular NVCP were detected by flow cytometry.

Detection of GFP Expression in Caco-2 Cells Incubated with NVCP

For detection by flow cytometry, Caco-2 cells were plated at 0.5 million/well in 6 well plates the day before the experiment. The next day, the media in each well was replaced with 1 ml of fresh complete culture media and then 0, 25, 50, or 75 μl of purified NVCP was added to each well. The cells were incubated for 48 hours. After that, the cells were washed and scraped from the wells and resuspended in PBS. Then GFP expression was detected by flow cytometry.

For detection by imaging, Caco-2 cells were plated at 20,000 cells/well in a 48-well tissue culture plate on Day 1. On Day 2, the cells were transfected with either empty parental plasmid or plasmid comprising Rep or RepA cDNA. On Day 3, 3 μg of NVCP in PBS was added to each well. Then the cells were incubated for 48 hours. On Day 5, GFP expression in each well was detected using the Evos Cell Imaging System (ThermoFisher).

For examining the inhibition activity of A10 protein on the replication of SARS-CoV-2 in Vero-E6 cells by VLP-delivered A10 gene, Vero-E6 cells were plated at 20,000 cells on day 1. Each well was transfected with 0.05 μg/well Rep and RepA on day 2. 200 μg/ml NVCP VLPs that encapsulate either A10 or GFP (negative control) gene construct were added to each well 5 hours after Rep/A transfection. On day 3, the cells were infected with 2000 pfu/well SARS-Cov2 WA strain. On day 4, Vero-E6 cells were fixed and stained with an antibody (CR3022) against the spike protein of SARS-CoV-2, followed by an HRP-conjugated goat anti-human antibody. The plate was then incubated with KPL Trueblue substrate (Seracare LifeScience) and imaged on an AID ELISpot Reader.

### EMBODIMENTS OF THE INVENTION

In some embodiments the disclosure relates to any of the following numbered paragraphs:
1. A virus-like particle (VLP) comprising a plant virus capsid protein and containing a replicon comprising a plant promoter operably linked to a heterologous polynucleotide, wherein the 3′ end of the heterologous polynucleotide is flanked by a geminiviral short intergenic region (SIR) sequence, and wherein the 5′ and 3′ ends of the replicon consist of a geminiviral long intergenic region (LIR) sequence.
2. The VLP of embodiment 1, wherein the VLP was produced in a plant cell.
3. The VLP of embodiment 1 or 2, wherein the plant virus capsid protein is a cowpea mosaic virus capsid protein or a tobacco mosaic virus capsid protein.
4. The VLP of any one of embodiments 1-3, wherein the heterologous polynucleotide encodes a protein.
5. The VLP of embodiment 4, wherein the protein is a protein-based pesticide, a protein-based means of herbicide resistance, a homing protein, or a gene editing reagent.
6. The VLP of embodiment 4 or 5, wherein the VLP expresses the protein encoded by the heterologous polynucleotide on its surface.
7. The VLP of any one of embodiments 1-3, wherein the heterologous polynucleotide encodes an RNA.
8. The VLP of embodiment 7, wherein the RNA is a small interfering RNA (siRNA), short hairpin RNA (shRNA), anti-sense RNA, microRNA (miRNA), or guide RNA (gRNA).
9. The VLP of any one of embodiments 1-8, further comprising a construct comprising a plant promoter operably linked to a polynucleotide encoding Rep and RepA.
10. The VLP of embodiment 9, wherein the replicon is replicated in plant cells.
11. A composition comprising the VLP of any one of the preceding embodiments and a carrier.
12. A method for producing a nucleic acid-loaded VLP, the method comprising:
   a) introducing into a plant cell a first construct comprising a first replicon that comprises a plant promoter operably linked to a first polynucleotide encoding plant virus capsid protein, wherein the 3′ end of the first polynucleotide is flanked by a geminiviral SIR sequence, and wherein the 5′ and 3′ ends of the first replicon consist of a geminiviral LIR sequence;
   b) introducing into the plant cell a second construct comprising a second replicon that comprises a plant promoter operably linked to a second, heterologous polynucleotide, wherein the 3′ end of the heterologous polynucleotide is flanked by a geminiviral SIR sequence, and wherein the 5′ and 3′ ends of the second replicon consist of a geminiviral LIR sequence;
   c) introducing a third construct comprising a plant promoter operably linked to a third polynucleotide encoding Rep and RepA; and
   d) harvesting the VLPs produced by the plant cell.
13. The method of embodiment 12, wherein the plant cell is part of a plant.
14. The method of embodiment 13, wherein the plant cell is part of a leaf of the plant.
15. The method of embodiment 13 or 14, wherein the plant is Nicotiana benthamiana.
16. The method of any one of embodiments 12-15, wherein one or more of the first construct, the second construct, and the third construct are integrated into the genome of the plant cell.
17. The method of any one of embodiments 12-16, wherein the plant virus capsid protein is cowpea mosaic virus capsid protein or a tobacco mosaic virus capsid protein.
18. The method of any one of embodiments 12-17, wherein two or more of the first construct, the second construct, the third construct are part of a single polynucleotide.
19. A method of using the VLP of any one of embodiments 1-10 or the composition of embodiment 11 to deliver the heterologous polynucleotide to a plant cell, the method comprising delivering the VLP to a plant.
20. The method of embodiment 19, wherein the heterologous polynucleotide encodes a gene product that modulates the expression of a gene in the plant.
21. The method of embodiment 20, wherein the gene product is a microRNA (miRNA) or a small interfering RNA (siRNA) and expression of the gene is repressed via RNA interference.
22. The method of embodiment 20, wherein the gene product is a gene editing reagent and the gene is edited.
23. The method of any one of embodiments 19-22, wherein the method increases the resistance of the plant to an herbicide, insect, pest, infection, extreme temperatures, or drought.
24. The method of any one of embodiments 19-23, wherein the plant is a crop, and wherein the method increases the yield of the crop or improves the taste of the edible portion of the crop.

What is claimed:

1. A virus-like particle (VLP) comprising a plant virus capsid protein and containing a replicon comprising a plant promoter operably linked to a heterologous polynucleotide, wherein the 3′ end of the heterologous polynucleotide is flanked by a geminiviral short intergenic region (SIR) sequence, and wherein the 5′ and 3′ ends of the replicon consist of a geminiviral long intergenic region (LIR) sequence.

2. The VLP of claim 1, wherein the VLP was produced in a plant cell.

3. The VLP of claim 1, wherein the plant virus capsid protein is a cowpea mosaic virus capsid protein or a tobacco mosaic virus capsid protein.

4. The VLP of claim 1, wherein the heterologous polynucleotide encodes a protein.

5. The VLP of claim 4, wherein the protein is a protein-based pesticide, a protein-based means of herbicide resistance, a homing protein, or a gene editing reagent.

6. The VLP of claim 4, wherein the VLP expresses the protein encoded by the heterologous polynucleotide on its surface.

7. The VLP of claim 1, wherein the heterologous polynucleotide encodes an RNA.

8. The VLP of claim 7, wherein the RNA is a small interfering RNA (siRNA), short hairpin RNA (shRNA), anti-sense RNA, microRNA (miRNA), or guide RNA (gRNA).

9. The VLP of claim 1, further comprising a construct comprising a plant promoter operably linked to a polynucleotide encoding Rep and RepA.

10. The VLP of claim 9, wherein the replicon is replicated in plant cells.

11. A composition comprising the VLP of claim 1 and a carrier.

12. A method for producing a nucleic acid-loaded VLP, the method comprising:
a) introducing into a plant cell a first construct comprising a first replicon that comprises a plant promoter operably linked to a first polynucleotide encoding plant virus capsid protein, wherein the 3′ end of the first polynucleotide is flanked by a geminiviral SIR sequence, and wherein the 5′ and 3′ ends of the first replicon consist of a geminiviral LIR sequence;
b) introducing into the plant cell a second construct comprising a second replicon that comprises a plant promoter operably linked to a second, heterologous polynucleotide, wherein the 3′ end of the heterologous polynucleotide is flanked by a geminiviral SIR sequence, and wherein the 5′ and 3′ ends of the second replicon consist of a geminiviral LIR sequence;
c) introducing a third construct comprising a plant promoter operably linked to a third polynucleotide encoding Rep and RepA; and
d) harvesting the VLPs produced by the plant cell.

13. The method of claim 12, wherein the plant cell is part of a plant.

14. The method of claim 13, wherein the plant cell is part of a leaf of the plant.

15. The method of claim 13, wherein the plant is Nicotiana benthamiana.

16. The method of claim 12, wherein one or more of the first construct, the second construct, and the third construct are integrated into the genome of the plant cell.

17. The method of claim 12, wherein the plant virus capsid protein is cowpea mosaic virus capsid protein or a tobacco mosaic virus capsid protein.

18. The method claim 12, wherein two or more of the first construct, the second construct, the third construct are part of a single polynucleotide.

19. A method of using the VLP of claim 1 to deliver the heterologous polynucleotide to a plant cell, the method comprising delivering the VLP to a plant.

20. The method of claim 19, wherein the heterologous polynucleotide encodes a gene product that modulates the expression of a gene in the plant.

21. The method of claim 20, wherein the gene product is a microRNA (miRNA) or a small interfering RNA (siRNA) and expression of the gene is repressed via RNA interference.

22. The method of claim 20, wherein the gene product is a gene editing reagent and the gene is edited.

23. (canceled)

24. The method of claim 19, wherein the plant is a crop, and wherein the method increases the yield of the crop or improves the taste of the edible portion of the crop.
